(19) United States
(12) Patent Application Publication
(10) Pub. No.: US 2003/0162980 A1
(43) Pub. Date: Aug. 28, 2003

(54) Method for producing 3,4-dihydro-2h-pyran

(76) Inventors: Hagen Weigl, Ladenburg (DE); Klaus Ebel, Lampertheim (DE); Joachim Simon, Mannheim (DE)

Correspondence Address: KEIL & WEINKAUF, 1350 CONNECTICUT AVENUE, N.W., WASHINGTON, DC, 20036, US

(21) Appl. No.: 10/343,018

(22) PCT Filed: Jul. 25, 2001
(86) PCT No.: PCT/EP01/08593

(51) Int. Cl. C07D 309/00

(52) U.S. Cl. 549/356

(57) ABSTRACT

A process for preparing 3,4-dihydro-2H-pyran by reacting tetrahydrofurfuryl alcohol over solid oxidic catalysts at temperatures of from 150 to 400° C. and pressures of from 0.001 to 50 bar, which comprises carrying out the reaction in the gas phase in a tube bundle reactor.

The present invention relates to a process for preparing 3,4-dihydro-2H-pyran by dehydrating rearrangement of tetrahydrofurfuryl alcohol over oxidic catalysts in a tube bundle reactor.

EP-A 691 337 discloses a process for preparing 3,4-dihydro-2H-pyran by converting tetrahydrofurfuryl alcohol in a fluidized bed. As well as high apparatus costs, this procedure in a fluidized bed requires high energy costs because of the gas quantity to be circulated and has a comparatively low space-time yield. A further disadvantage is that the high mechanical stress on the catalyst particles leads to attrition and accordingly to efflux of fine catalyst material in the reaction effluent.

The preparation of 3,4-dihydro-2H-pyran by reacting tetrahydrofurfuryl alcohol in a solid bed at 375° C. over activated aluminum oxide is disclosed in J. Am. Chem. Soc., 68 (1946), pages 1646 to 1648. Noncompliance with the complicated start-up procedure leads to temperature spikes of up to 525° C. and to deactivation of the catalyst.

Organic Syntheses, Col. Vol. 3, pages 276 to 277, 1955 discloses a process for preparing 3,4-dihydro-2H-pyran by reacting tetrahydrofurfuryl alcohol in a fixed bed reactor. The onstream times of the activated aluminum oxide used are reported as 12 to 16 hours. On the catalyst, brown, tarry deposits are formed.

GB-A-10 17 313 suggests preparing 3,4-dihydro-2H-pyran from tetrahydrofurfuryl alcohol using vanadium oxide or molybdenum oxide on activated aluminum oxide catalysts. The yields are up to 76%.

GB 858 626 also discloses the conversion of tetrahydrofurfuryl alcohol over titanium dioxide/aluminum oxide catalysts. The conversion is initially 97%, but after just 20 days is only an unsatisfactory 70%. The attainable catalyst onstream times are unsatisfactory.

It is an object of the present invention to remedy the abovementioned disadvantages.

We have found that this object is achieved by a novel and improved process for preparing 3,4-dihydro-2H-pyran by reacting tetrahydrofurfuryl alcohol over solid oxidic catalysts at temperatures of from 150 to 400° C. and pressures of from 0.001 to 50 bar, which comprises carrying out the reaction in the gas phase in a tube bundle reactor.

A tube bundle reactor consists of at least one reactor tube which is surrounded by a heat transfer medium for heating and/or cooling. In general, the tube bundle reactors used industrially comprise from more than three to tens of thousands of parallel reactor tubes. If more than one individual tube bundle reactor (in the sense of tube bundle reactor apparatus) is attached in parallel, these can be regarded as the equivalent of a tube bundle reactor and are included in the term tube bundle reactor in the following.

If the tube bundle reactor unit consists of more than one tube bundle reactor, for example, two, three, four or more, these are attached in series. In general, the tube bundle reactors are attached in direct succession, i.e. the exit stream of one tube bundle reactor is passed directly into the entrance of the following reactor. However, it is also possible to transfer mass and/or energy to or from the two tube bundle reactors. For example, a portion of the gas stream or a component thereof may be withdrawn or a further gas stream may be added or the gas stream present may be passed through a heat exchanger.

The tube bundle reactor unit may further comprise one or more preheating zones which heat the entering gas mixture. A preheating zone integrated into a tube bundle reactor may be realized, for example, by reactor tubes filled with inert material which are likewise surrounded by a heat transfer medium. In principle, useful inert materials include all shaped bodies which are chemically inert, i.e. do not induce or catalyze any heterogeneously catalytic reaction and have a maximum pressure drop below the maximum tolerable plant-specific value in each case. Examples of useful materials include oxidic materials, such as Al₂O₃ or SiC, or metallic materials, such as stainless steel. Examples of shaped bodies include spheres, tablets, hollow cylinders, rings, trilobes, tristars, wagon wheels, extrudates and randomly shaped bodies.

The process according to the invention in a tube bundle reactor advantageously facilitates substantially isothermal reaction. Substantially isothermal means that the maximum temperature difference between the hottest and the coldest reaction zones is 5° C. at most. The isothermal procedure facilitates long onstream times of the catalysts used.

The process according to the invention can be carried out as follows:

Tetrahydrofurfuryl alcohol is passed in gaseous form, preferably with an inert carrier gas such as nitrogen or a noble gas such as argon, preferably nitrogen, at a temperature of from 150 to 400° C., preferably from 200 to 350° C., more preferably at from 250 to 300° C., through the tube bundle reactor packed with catalyst.

Preference is given to substantially recycling the inert gas and only supplementing it with small quantities of fresh gas. The reaction pressure may be varied within wide limits and is generally from 0.001 to 50 bar, preferably from 0.01 to 10 bar, more preferably from atmospheric pressure to 1.5 bar. The gas mixture leaving the tube bundle reactor is condensed, and the organic phase removed and fractionated. The 3,4-dihydro-2H-pyran may be subjected to further purification by distillation.

Useful solid oxidic catalysts include oxides of groups IIa, IIIa, IIb, IIIb, IVb, Vb, VIb and VIIb of the Periodic Table, of iron, cobalt, nickel, cerium, praseodymium or mixtures thereof, preferably oxides of magnesium, calcium, aluminum, zinc, titanium, zirconium, vanadium, chromium, molybdenum, manganese, iron, cobalt, nickel, cerium or mixtures thereof, more preferably oxides of magnesium, calcium, aluminum, zinc, titanium, zirconium, manganese, iron, cobalt, nickel or mixtures thereof, in particular aluminum oxide-containing, more preferably gamma-aluminum oxide-containing, mixtures which have a gamma-aluminum oxide content in the oxide mixture of up to 100% by weight. These solid oxidic catalysts may optionally be saturated with phosphoric acid. The catalysts usable according to the invention may be present as unsupported catalysts or supported on suitable support materials, for example, aluminum oxide, titanium oxide, zirconium oxide, magnesium oxide or mixtures thereof, although the use of unsupported catalysts is preferred. The catalytically active oxides are applied to the support material in a manner known per se.

The catalyst hourly space velocity in the process according to 40 the invention is generally from 0.005 to 0.5 kg/l_{catalyst} h, preferably from 0.05 to 0.2 kg/l_{catalyst} h.

The process according to the invention may be operated either continuously or batchwise, although a continuous procedure is preferred.

In comparison to the known processes, the process according to the invention delivers 3,4-dihydro-2H-pyran in a simpler and more economical way. The process may be operated isothermally to facilitate long onstream times of the catalysts used. In 5 addition, 3,4-dihydro-2H-pyran is obtained in yields of over 80% and purities of over 90%.

3,4-Dihydro-2H-pyran finds a broad range of application as a valuable protecting group for alcohols in preparing industrial organic specialty chemicals.

### EXAMPLES

### Example 1

The experimental plant was equipped with a feed unit which ensures controlled feed of tetrahydrofurfuryl alcohol and a reactor tube. The replacement of a tube bundle reactor by a reactor tube is very feasible on a laboratory or pilot plant scale, as long as the measurements of the reactor tube are in the region of those of an industrial reactor tube. The plant was operated in “straight pass”, i.e. without recirculation.

The tube bundle reactor unit consisted of a reactor tube having a length of 0.8 m and an internal diameter of 30 mm. Within the reactor tube, a multi-thermoelement having temperature measurement points was located in a protective tube. The reactor tube was surrounded by a heatable heat transfer circuit. Tube bundle reactor flow was downward. The heat transfer medium used was a heat transfer oil.

51 g of tetrahydrofurfuryl alcohol per hour were evaporated in the feed unit and passed with 50 l (STP) of nitrogen through the tube bundle reactor tube heated to 250° C. The tube bundle reactor tube was filled with 250 ml (150 g) of gamma-aluminum oxide. The reaction gases were then condensed and the phases separated. 23.5 g (95% by weight) per hour of 3,4-dihydro-2H-pyran of 95 GC-area % purity (95 area percent by gas chromatography) were obtained. The tetrahydropyran content was below 1% by weight. The activity of the catalyst was unchanged after 1360 hours. The conversion was 96%.

### Example 2

Example 1 was repeated, except that 33 g per hour of tetrahydrofurfuryl alcohol were used. On average after a running time of 3520 hours, 23.2 g per hour of 3,4-dihydro-2H-pyran of 94 GC-area % purity (94 area percent by gas chromatography) are obtained. The conversion fell in the long running time to a minimum of 90%, but could be brought back up to 95° C. by increasing the temperature by 5° C. After 3520 hours, the activity of the catalyst was virtually unchanged.

### Example 3

The experimental plant was equipped with a feed unit and a tube bundle reactor. The plant was operated by the recycle gas method.

The tube bundle reactor consisted of 30 reactor tubes of 1.7 m in length and 30 mm internal diameter. Within five of these reactor tubes, a multi-thermoelement having in each case three temperature measurement points was located in a protective tube. The reactor tubes were surrounded by heatable heat transfer circuits. Tube bundle reactor flow was downward. 5% per hour of the recycle gas was replaced by fresh gas. The heat transfer medium used was a salt melt.

3.2 kg (3.2 l) per hour of tetrahydrofurfuryl alcohol were evaporated in the feed unit and passed with 2800 l (STP) of nitrogen through the tube bundle reactor heated to 250° C. The tube bundle reactor was filled with 34 l (18 kg) of gamma-aluminum oxide. The reaction gases were then condensed and the phases separated. 2.3 kg (calc. 100%) per hour were obtained, corresponding to a yield of 87% by weight of 3,4-dihydro-2H-pyran.

We claim:

1. A process for preparing 3,4-dihydro-2H-pyran by reacting tetrahydrofurfuryl alcohol over solid oxidic catalysts at temperatures of from 150 to 400° C. and pressures of from 0.001 to 50 bar, which comprises carrying out the reaction in the gas phase in a tube bundle reactor.

2. A process for preparing 3,4-dihydro-2H-pyran as claimed in claim 1, wherein the solid oxidic catalysts used are oxides of groups IIa, IIIa, IIb, IIIb, IVb, Vb, VIb or VIIb of the Periodic Table, of iron, cobalt, nickel, cerium or praseodymium, or mixtures thereof.

3. A process for preparing 3,4-dihydro-2H-pyran as claimed in claim 1, wherein the solid oxidic catalysts used are oxides of magnesium, calcium, aluminum, zinc, titanium, zirconium, vanadium, chromium, molybdenum, manganese, iron, cobalt, nickel, cerium or mixtures thereof.

4. A process for preparing 3,4-dihydro-2H-pyran as claimed in claim 1, wherein the solid oxidic catalysts used are oxides of magnesium, calcium, aluminum, zinc, titanium, zirconium, manganese, iron, cobalt, nickel or mixtures thereof.

5. A process for preparing 3,4-dihydro-2H-pyran as claimed in claim 1, wherein the solid oxidic catalysts used are aluminum oxide.

6. A process for preparing 3,4-dihydro-2H-pyran as claimed in claim 1, wherein the reaction is carried out at temperatures of from 250 to 300° C.

7. A process for preparing 3,4-dihydro-2H-pyran as claimed in claim 1, wherein an inert carrier gas is substantially recycled.
